(19) United States
(12) Patent Application Publication
(10) Pub. No.: US 2024/0197620 A1
(43) Pub. Date: Jun. 20, 2024

(54) SKIN PENETRATION ENHANCER FOR ROYAL JELLY

(71) Applicant: Yamada Bee Company, Inc., Tomata-gun (JP)

(72) Inventors: Masayuki YAGI, Tomata-gun (JP); Yuka MAEDA, Tomata-gun (JP)

(21) Appl. No.: 18/384,050

(22) Filed: Oct. 26, 2023

(30) Foreign Application Priority Data
Dec. 16, 2022 (JP) 2022-201184

(51) Int. Cl. A61K 8/98; A61K 8/81; A61Q 19/00

(52) U.S. Cl. CPC A61K 8/988; A61K 8/8141; A61K 8/8147; A61Q 19/00; A61K 2800/10

(57) ABSTRACT

A method for enhancing penetration of a royal jelly into a skin includes blending an (acrylates/(C10-30) alkyl acrylate) crosspolymer and/or an (ammonium acryloyldimethyltaurate/methacrylic acid beheneth-25) crosspolymer into a cosmetic composition including the royal jelly. A method for enhancing penetration of a royal jelly into a skin also includes applying a cosmetic composition including an (acrylates/(C10-30) alkyl acrylate) crosspolymer and/or an (ammonium acryloyldimethyltaurate/methacrylic acid beheneth-25) crosspolymer and the royal jelly on the skin. A method for producing a cosmetic composition including a royal jelly includes blending an (acrylates/(C10-30) alkyl acrylate) crosspolymer and/or an (ammonium acryloyldimethyltaurate/methacrylic acid beheneth-25) crosspolymer into the cosmetic composition for enhancing penetration of the royal jelly into a skin.

### CROSS-REFERENCE TO RELATED APPLICATION

The present invention claims priority under 35 U.S.C. §119 to Japanese Application, 2022-201184 filed on Dec. 16, 2022, the entire contents of which being incorporated herein by reference.

### TECHNICAL FIELD

The present invention relates to a skin penetration enhancer for royal jelly.

### BACKGROUND

It is known that royal jelly exhibits, when applied on the skin, a skin aging prevention effect, a cosmetic effect leading to moist and firm skin, and the like (for example, Japanese Unexamined Patent Publication No. 2007-204418). These cosmetic effects are believed to be brought by fatty acids characteristic of royal jelly, such as 10-hydroxy-2-decenoic acid in particular. Therefore, in order to have the cosmetic effect of royal jelly fully exhibited, particularly the skin penetrability of the fatty acids characteristic of royal jelly is important. Japanese Unexamined Patent Publication No. 2015-007123 discloses a skin preparation for external use including a royal jelly extract as a skin activating component, and it is described that as this skin preparation for external use stably retains moisture on the skin for a long time period, the penetrability of the skin activating component into the skin is elevated.

### SUMMARY

It is an object of the present invention to provide a skin penetration enhancer for royal jelly.

The inventors of the present invention conducted a thorough investigation in order to achieve the above-described object, and as a result, the inventors newly found that an (acrylates/(C10-30) alkyl acrylate) crosspolymer and an (ammonium acryloyldimethyltaurate/methacrylic acid beheneth-25) crosspolymer used as a cosmetic additive enhance the penetration of royal jelly into the skin, thus completing the present invention. As used herein, crosspolymers refer to copolymers that are crosslinked.

That is, the present invention relates to, for example, the following inventions.
[1] A skin penetration enhancer for a royal jelly, comprising an (acrylates/(C10-30) alkyl acrylate) crosspolymer and/or an (ammonium acryloyldimethyltaurate/methacrylic acid beheneth-25) crosspolymer as an active ingredient.
[2] The skin penetration enhancer according to [1], wherein the royal jelly comprises saturated fatty acids and/or unsaturated fatty acids.
[3] The skin penetration enhancer according to [2], wherein the saturated fatty acids comprise 10-hydroxydecanoic acid.
[4] The skin penetration enhancer according to [2], wherein the unsaturated fatty acids comprise at least one of 10-hydroxy-2-decenoic acid, decenedioic acid, or sebacic acid.
[5] A cosmetic composition comprising the skin penetration enhancer according to any one of [1] to [4] and a royal jelly.
[6] A method for enhancing penetration of a royal jelly into a skin, the method comprising blending the skin penetration enhancer according to any one of [1] to [4] into a cosmetic composition comprising the royal jelly.
[7] A method for enhancing penetration of a royal jelly into a skin, the method comprising applying the cosmetic composition according to [5] on the skin.
[8] A method for enhancing penetration of a royal jelly into a skin, the method comprising blending an (acrylates/(C10-30) alkyl acrylate) crosspolymer and/or an (ammonium acryloyldimethyltaurate/methacrylic acid beheneth-25) crosspolymer into a cosmetic composition comprising the royal jelly.
[9] The method according to [8], wherein the royal jelly comprises saturated fatty acids and/or unsaturated fatty acids.
[10] The method according to [9], wherein the saturated fatty acids comprise 10-hydroxydecanoic acid.
[11] The method according to [9], wherein the unsaturated fatty acids comprise at least one of 10-hydroxy-2-decenoic acid, decenedioic acid, or sebacic acid.
[12] A method for enhancing penetration of a royal jelly into a skin, the method comprising applying a cosmetic composition comprising an (acrylates/(C10-30) alkyl acrylate) crosspolymer and/or an (ammonium acryloyldimethyltaurate/methacrylic acid beheneth-25) crosspolymer and the royal jelly on the skin.
[13] The method according to [12], wherein the royal jelly comprises saturated fatty acids and/or unsaturated fatty acids.
[14] The method according to [13], wherein the saturated fatty acids comprise 10-hydroxydecanoic acid.
[15] The method according to [13], wherein the unsaturated fatty acids comprise at least one of 10-hydroxy-2-decenoic acid, decenedioic acid, or sebacic acid.
[16] The method according to any one of to [15], wherein a content of the (acrylates/(C10-30) alkyl acrylate) crosspolymer in the cosmetic composition is 0.01% by mass or more and 20% by mass or less, with respect to the total amount of the cosmetic composition.
[17] The method according to any one of to [16], wherein a content of the (ammonium acryloyldimethyltaurate/methacrylic acid beheneth-25) crosspolymer in the cosmetic composition is 0.1% by mass or more and 20% by mass or less, with respect to the total amount of the cosmetic composition.
[18] The method according to any one of to [17], wherein a content of the royal jelly in the cosmetic composition is 0.1% by mass or more and 20% by mass or less, with respect to the total amount of the cosmetic composition.
[19] A method for producing a cosmetic composition comprising a royal jelly, the method comprising blending an (acrylates/(C10-30) alkyl acrylate) crosspolymer and/or an (ammonium acryloyldimethyltaurate/methacrylic acid beheneth-25) crosspolymer into the cosmetic composition for enhancing penetration of the royal jelly into a skin.
[20] The method according to [19], wherein the royal jelly comprises saturated fatty acids and/or unsaturated fatty acids.
[21] The method according to [20], wherein the saturated fatty acids comprise 10-hydroxydecanoic acid.
[22] The method according to [20], wherein the unsaturated fatty acids comprise at least one of 10-hydroxy-2-decenoic acid, decenedioic acid, or sebacic acid.

According to the present invention, a cosmetic effect of a cosmetic composition including royal jelly can be heightened by enhancing the penetration of royal jelly into the skin.

### BRIEF DESCRIPTION OF THE DRAWINGS

FIG. 1 shows the results of imaging mass spectrometry (MS) showing the penetrability of 10-hydroxy-2-decenoic acid into the skin.

FIG. 2 shows the results of imaging MS showing the penetrability of 10-hydroxydecanoic acid into the skin.

### DETAILED DESCRIPTION

Embodiments for carrying out the present invention will be described in detail

below. However, the present invention is not intended to be limited to the following embodiments.

The skin penetration enhancer for royal jelly of the present invention includes an (acrylates-(C10-30) alkyl acrylate) crosspolymer and/or an (ammonium acryloyldimethyltaurate/methacry lic acid beheneth-25) crosspolymer as an active ingredient.

The (acrylates/(C10-30) alkyl acrylate) crosspolymer is a product obtained by crosslinking a copolymer of an alkyl acrylate and one or more monomers consisting of acrylic acid, methacrylic acid, or simple esters thereof, with allyl ether of sucrose or allyl ether of pentaerythritol. Here, the alkyl acrylate is specifically an alkyl acrylate having an alkyl with 10 to 30 carbon atoms. As the (acrylates/(C10-30) alkyl acrylate) crosspolymer, a commercially available (acrylates/(C10-30) alkyl acrylate) crosspolymer (for example, PEMULEN (trademark) TR-1) may be used.

The (ammonium acryloyldimethyltaurate/methacrylic acid beheneth-25) crosspolymer is a copolymer an ammonium salt of acryloyldimethyltaurine and methacrylic acid ester of beheneth-25. Beheneth-25 is polyethylene glycol ether of behenyl alcohol.

The (acrylates/(C10-30) alkyl acrylate) crosspolymer and the (ammonium acryloyldimethyltaurate/methacrylic acid beheneth-25) crosspolymer are cosmetic components known as a thickener or an emulsion stabilizer; however, the effect of enhancing the penetration of royal jelly into the skin has not been known yet.

The enhancement of penetration of royal jelly into the skin means that the components included in royal jelly, particularly active ingredients having cosmetic effects, for example, components such as saturated fatty acids and unsaturated fatty acids, penetrate more deeply into the horny layer of the skin as compared to the case where a skin penetration enhancer is not included. Here, examples of the saturated fatty acids include 10-hydroxydecanoic acid, 8-hydroxyoctanoic acid, 12-hydroxydodecanoic acid, 3,10-dihydroxydecanoic acid, and capric acid, and examples of the unsaturated fatty acids include 10-hydroxy-2-decenoic acid, decenedioic acid, sebacic acid, and trans-2-decenoic acid. Preferred examples of the saturated fatty acids include 10-hydroxydecanoic acid, and preferred examples of the unsaturated fatty acids include 10-hydroxy-2-decenoic acid, decenedioic acid, and sebacic acid.

The enhancement of penetration of royal jelly into the skin can be evaluated by analyzing the components included in royal jelly, particularly components such as saturated fatty acids and unsaturated fatty acids, for example, by the method described in the Examples. Specifically, a method of applying a composition including royal jelly and a skin penetration enhancer on the skin, collecting a horny layer sample after a predetermined time (for example, after 30 minutes, after 1 hour, or after 2 hours) by performing a tape stripping test, and detecting royal jelly that has attached to the tape, may be mentioned.

The method for detecting the royal jelly components that have penetrated into the horny layer is not particularly limited, and for example, the royal jelly components may be detected by imaging using a mass spectrometer as will be described in the Examples of the present application.

### Royal Jelly

Royal jelly is a milky white gelatinous substance made by 3- to 12-day-old worker bees among honeybees through mixing of a secretion secreted from the hypopharyngeal gland and mandibular gland. Examples of major biologically active components in royal jelly include organic acids such as the saturated fatty acids and unsaturated fatty acids unique to royal jelly, a protein, an amino acid, a peptide, a lipid, sugars, vitamins such as vitamin Bs, a folic acid, a nicotinic acid, and a pantothenic acid, and various types of minerals.

In the present specification, the royal jelly may be, for example, raw royal jelly or may be a royal jelly treated product prepared by subjecting raw royal jelly to treatment.

Raw royal jelly can be obtained for example as a beekeeping product in accordance with a conventional method. A place of production of royal jelly is not limited, and may be any of European countries, Oceanian countries, the U.S.A., Brazil, Japan, China, and other Asian countries and the like. The type of honey bees utilized for collecting raw royal jelly is not limited. Examples of raw royal jelly include frozen royal jelly prepared by cryopreservation of collected royal jelly.

Examples of treated royal jelly include: a royal jelly concentrates or dilutions prepared by concentrating or diluting raw royal jelly: a dried royal jelly powder prepared by drying and powderizing raw royal jelly: a royal jelly extract prepared by extracting royal jelly with water or a solvent such as ethanol: and an enzymatically treated royal jelly prepared by treating royal jelly with a protease. The treated royal jelly may be a royal jelly having been subjected to a plurality of treatments. For example, the royal jelly may be an enzymatically treated royal jelly powder obtained by enzymatically treated and powderization.

The royal jelly concentrate can be obtained by, for example, removing moisture content from raw royal jelly. The royal jelly dilution can be obtained by, for example, adding water to raw royal jelly.

The royal jelly powder can be obtained by, for example, powderizing raw royal jelly through a method known in this technical field, such as freeze-drying or spray drying and the like. As a drying method, any known method that is employed in general food processing, such as natural drying, e.g., ventilation drying and solar drying, forced drying that involves heating with electricity or the like for drying, and freeze-drying can be used. Preferably freeze-drying is used. Note that the time for drying is not particularly limited, and example thereof can include about 3 days in the case of natural drying such as ventilation drying or solar drying, and about 1 to 3 days at about 50° C. in the case of forced drying via heating with electricity or the like. Usually, drying is preferably performed in such a manner that the water content is 10% by mass or less, and preferably 5% by mass or less. Note that if it is difficult to lower the water content to 10% by mass or less as in the case of natural drying such as ventilation drying or solar drying, the dried product may be further subjected to a treatment with a freeze-dryer for further lowing the water content. In addition, royal jelly powder may be obtained through pulverization by using a pulverizer (for example, a pin mill, a hammer mill, a ball mill, or a jet mill) to obtain a royal jelly powder.

The royal jelly extract can be obtained by, for example, extracting raw royal jelly, a royal jelly powder, or an enzymatically treated royal jelly by using, as a solvent such as water, ethanol, methanol, propanol, or acetone as an extracting solvent. The extraction time can be appropriately set according to the form of royal jelly used as the starting material, the type and amount of the solvent, the temperature, and the stirring conditions in the extraction, etc. After the extraction, a solid matter may be removed by filtration, centrifugation, or the like. The extraction solution may be used as it is, or the solution may be used as a concentrated solution or a dried powder by removing the solvent from the solution. The royal jelly extract may be an aqueous extract of royal jelly extracted with water or may be an ethanol extract of royal jelly extract with ethanol.

The enzymatically treated royal jelly can be obtained by, for example, treating raw royal jelly or a royal jelly powder with a protease. Examples of protease is preferably selected from the group consisting of an enzyme having at least endopeptidase action, an enzyme having at least exopeptidase action, and an enzyme having both endopeptidase action and exopeptidase action. In particular, protease is preferably a peptidase simultaneously having both endopeptidase action and exopeptidase action. Enzymatic treatment using such peptidase can lower the molecular weight of a protein with one-step enzymatic treatment, and thus is advantageous in that the operation is simple, as well as the disappearance of and a significant reduction in the bioactivity of useful components contained in royal jelly can be prevented.

Preferred examples of the enzyme having endopeptidase activity can include Bacillus subtilis-produced peptidase (trade name: ORIENTASE 22BF, NUCLEICIN), Bacillus licheniformis-produced peptidase (trade name: ALCALASE), Bacillus stearothermophilus-produces peptidase (trade name: PROTEASE Bacillus amyloliquefaciens-produced peptidase (trade name: NEUTRASE), and the genus Bacillus-produced peptidase (trade name: PROTAMEX).

Preferred examples of the enzyme having exopeptidase activity can include Aspergillus oryzae-produced peptidase (trade name: UMAMIZYME G, PROMOD 192P, PROMOD 194P, SUMIZYME FLAP), Aspergillus sojae-produced peptidase (trade name: STERNZYME B15024), the genus Aspergillus-produced peptidase (trade name: KOKULASE P), and Rhizopus oryzae-produced peptidase (trade name: PEPTIDASE R).

Preferred examples of the enzyme having both exopeptidase activity and endopeptidase activity can include Aspergillus oryzae-produced peptidase (trade name: SUMIZYME LP-G, PROTEAX, FLAVOURZYME, PROTEASE A), Streptomyces griseus-produced peptidase (trade name: ACTINASE AS), and Aspergillus melleus-produced peptidase (trade name: PROTEASE P).

The reaction conditions (the amount of protease to be used, temperature and pH at the time of the reaction, reaction time, etc.) when treating raw royal jelly or a royal jelly powder is treated with protease may be appropriately set, depending on the type of protease used, or the like.

The royal jelly is preferably an enzymatically treated royal jelly or a royal jelly extract, and particularly preferably an enzymatically treated royal jelly. It is preferable that the enzymatically treated royal jelly is obtained by treating royal jelly with an enzyme having both the exopeptidase activity and the endopeptidase activity.

Regarding the royal jelly, a commercially available one may be used. Specific examples of the royal jelly include, Royal Jelly FD Powder (Nakahara Co., Ltd.), Royal Jelly Extract SF (MATSUURA YAKUGYO CO., LTD.), Deproteinized Royal Jelly Powder F (MARUZEN PHARMACEUTICALS CO., LTD.), and Deproteinized Royal Jelly Extract (API Co., Ltd.).

The royal jelly is not particularly limited as long as it has, for example, a skin aging prevention effect and a cosmetic effect leading to moist and firm skin; however, it is preferable that the royal jelly includes saturated fatty acids and/or unsaturated fatty acids, it is preferable that the saturated fatty acids include 10-hydroxy-2-decenoic acid, and it is preferable that the unsaturated fatty acids include at least one of 10-hydroxydecanoic acid, decenedioic acid, or sebacic acid. The skin penetration enhancer of the present invention can be used for a skin preparation for external use, a cosmetic composition, and the like, all of which contain royal jelly.

### Cosmetic Composition

The cosmetic composition of the present invention includes the skin penetration enhancer for royal jelly of the present invention, and royal jelly.

The content of the (acrylates/(C10-30) alkyl acrylate) crosspolymer in the cosmetic composition may be any amount (effective amount) that can exhibit the skin penetration enhancing action for royal jelly, and for example, the content may be 0.01% by mass or more, 0.1% by mass or more, 0.3% by mass or more, 0.4% by mass or more, 0.5% by mass or more, 1% by mass or more, 2% by mass or more, 3% by mass or more, or 5% by mass or more, and may be 20% by mass or less, 15% by mass or less, 10% by mass or less, 8% by mass or less, 5% by mass or less, 3% by mass or less, 2% by mass or less, 1% by mass or less, or 0.5% by mass or less, with respect to the total amount of the cosmetic composition.

The content of the (ammonium acryloyldimethyltaurate/methacrylic acid beheneth-25) crosspolymer in the cosmetic composition may be any amount (effective amount) that can exhibit the skin penetration enhancing action for royal jelly, and for example, the content may be 0.1% by mass or more, 0.3% by mass or more, 0.5% by mass or more, 0.8% by mass or more, 1% by mass or more, 2% by mass or more, 3% by mass or more, or 5% by mass or more, and may be 20% by mass or less, 15% by mass or less, 10% by mass or less, 8% by mass or less, 5% by mass or less, 3% by mass or less, 2% by mass or less, 1% by mass or less, or 0.5% by mass or less, with respect to the total amount of the cosmetic composition. When the cosmetic composition includes both the (acrylates/(C10-30) alkyl acrylate) crosspolymer and the (ammonium acryloyldimethyltaurate/methacrylic acid beheneth-25) crosspolymer, the total content may be within the above-described range of the content.

The content of royal jelly in the cosmetic composition is not particularly limited as long as the content is in the range that allows royal jelly to exhibit a cosmetic effect, and for example, the content may be 0.1% by mass or more, 0.5% by mass or more, 1% by mass or more, 1.5% by mass or more, 2% by mass or more, 3% by mass or more, 5% by mass or more, 8% by mass or more, 10% by mass or more, or 15% by mass or more, and may be 20% by mass or less, 15% by mass or less, 10% by mass or less, 8% by mass or less, 5% by mass or less, 3% by mass or less, 2% by mass or less, 1% by mass or less, or 0.5% by mass or less, with respect to the total amount of the cosmetic composition.

The cosmetic composition may be in the form of any of a solid, a liquid, a paste, and the like. The cosmetic product may be a medicinal cosmetic product (that is, a quasi-drug). The cosmetic composition includes all kinds of cosmetic compositions that can be applied to sites such as skin, mucous membrane, body hair, head hair, scalp, nails, teeth, facial skin, and lips of animals (particularly, humans). Among these, from the viewpoint of enhancing the penetration of royal jelly, the cosmetic composition is preferably a cosmetic composition that is applied to the skin of the whole body, for example, face, scalp, and lips.

The cosmetic composition may include cosmetically acceptable components, in addition to the skin penetration enhancer of the present invention and royal jelly. Examples of the cosmetically acceptable components include a whitening agent, a moisturizer, an antioxidant, an oily component, an ultraviolet absorber, a surfactant, a thickener, an alcohol, a powder component, a color material, an aqueous component, water, and various skin nutrients.

The dosage form of the cosmetic composition may be, for example, a solubilized system, an emulsified system, a powdered system, an oily liquid system, a gel system, an ointment system, an aerosol system, a water-oil bilayer system, or a water-oil-powder trilayer system. The cosmetic product may be, for example, a basic skin care product such as a facial cleanser, a skin lotion, an emulsion, a cream, a gel, an essence, a beauty serum, a pack, a mask, a mist, or a UV-blocking cosmetic product: a makeup cosmetic product such as a foundation, a lipstick, a cheek rouge, an eyeshadow, an eyeliner, or a mascara: a facial cleanser, a massage agent, a cleansing agent, an after-shave lotion, a pre-shave lotion, a shaving cream, a body soap, a soap, a shampoo, a hair conditioner, a hair treatment, a hair styling material, a hair tonic agent, a hair mist, a hair foam, a hair liquid, a hair gel, a hair spray, a hair growth promoter, an antiperspirant, a bath additive, a mouthwash, an oral cosmetic product, a toothpaste, a hand cream, or a hand soap.

The production method for the above-described cosmetic composition is not particularly limited, and any known method can be appropriately adopted. For example, a cosmetic composition to be used for the above-described purpose can be obtained by mixing the above-described skin penetration enhancer and royal jelly into an intermediate product or a final product obtained in the production process for a cosmetic product.

The method for producing the cosmetic composition including the royal jelly according to an embodiment is the method including blending the (acrylates/(C10-30) alkyl acrylate) crosspolymer and/or the (ammonium acryloyldimethyltaurate/methacrylic acid beheneth-25) crosspolymer into the cosmetic composition for enhancing penetration of the royal jelly into the skin. The royal jelly may include saturated fatty acids and/or unsaturated fatty acids, it is preferable that the saturated fatty acids include 10-hydroxy-2-decenoic acid, and it is preferable that the unsaturated fatty acids include at least one of 10-hydroxydecanoic acid, decenedioic acid, or sebacic acid.

### Method for Enhancing Penetration of Royal Jelly Into Skin

The method for enhancing the penetration of royal jelly into the skin according to an embodiment includes blending the skin penetration enhancer of the present invention into a cosmetic composition including royal jelly. By blending the skin penetration enhancer of the present invention into a cosmetic composition including royal jelly, when the resulting cosmetic composition is applied on the skin, the penetration of the royal jelly included in the cosmetic composition into the skin is enhanced due to the presence of the skin penetration enhancer of the present invention, as compared with a cosmetic composition that does not include the skin penetration enhancer of the present invention.

The method for enhancing the penetration of the royal jelly into the skin according to another embodiment is the method includes blending the (acrylates/(C10-30) alkyl acrylate) crosspolymer and/or the (ammonium acryloyldimethyltaurate/methacrylic acid beheneth-25) crosspolymer into the cosmetic composition comprising the royal jelly for enhancing penetration of the royal jelly into the skin. The royal jelly may include saturated fatty acids and/or unsaturated fatty acids, it is preferable that the saturated fatty acids include 10-hydroxy-2-decenoic acid, and it is preferable that the unsaturated fatty acids include at least one of 10-hydroxydecanoic acid, decenedioic acid, or sebacic acid.

A method for enhancing the penetration of royal jelly into the skin according to a further embodiment includes applying the cosmetic composition of the present invention on the skin. By applying the cosmetic composition of the present invention on the skin, the penetration of the royal jelly included in the cosmetic composition into the skin is enhanced due to the presence of the skin penetration enhancer of the present invention, as compared with a cosmetic composition that does not include the skin penetration enhancer of the present invention.

The method for enhancing the penetration of the royal jelly into the skin according to a further other embodiment is the method includes applying the cosmetic composition including the (acrylates/(C10-30) alkyl acrylate) crosspolymer and/or the (ammonium acryloyldimethyltaurate/methacrylic acid beheneth-25) crosspolymer and the royal jelly on the skin. The royal jelly may include saturated fatty acids and/or unsaturated fatty acids, it is preferable that the saturated fatty acids include 10-hydroxy-2-decenoic acid, and it is preferable that the unsaturated fatty acids include at least one of 10-hydroxydecanoic acid, decenedioic acid, or sebacic acid.

### EXAMPLES

Hereinafter, Examples will be described below in order to describe the present application more specifically. However, the present application is not intended to be limited to the following Examples.

### Preparation of Enzymatically Treated Royal Jelly Extract

An enzymatically treated royal jelly extract was prepared by treating raw royal jelly according to the method described in WO 2020/196065 A1. Specifically, 400 ml of ion-exchanged water was added to 474 g of raw royal jelly, and the mixture was stirred until the mixture became uniform to prepare a royal jelly diluted solution. A 2 N aqueous solution of NaOH was added thereto to adjust the pH of the royal jelly diluted solution to 7.8. Next, a solution obtained by dissolving 4.75 g of PROTEAX (Amano Enzyme Inc.), which has both endopeptidase action and exopeptidase action, in 20 mL of ion-exchanged water was added to the royal jelly diluted solution, and ion-exchanged water was further added thereto so that the total amount reached 980 g. The reaction mixture was allowed to react at 50° C. (constant temperature water tank) for 2 hours while being stirred with a propeller to perform hydrolysis. The temperature of the constant temperature water tank was raised to 80° C., and the mixture was stirred for 15 minutes to deactivate the protease. The obtained enzymatically treated royal jelly solution was filtered through a filter having a pore size of 3 μm, and an enzymatically treated royal jelly solution was obtained. Butylene glycol, pentylene glycol, phenoxyethanol, and purified water were added thereto so that the concentration of the enzymatically treated royal jelly solution was 2.5% by weight, and an enzymatically treated royal jelly extract was obtained.

### Preparation of Cosmetic Composition of Formulation Example 1

The enzymatically treated royal jelly extract, an (acrylates/(C10-30) alkyl acrylate) crosspolymer as a skin penetration enhancer, and other raw materials described in Table 1 were mixed at predetermined proportions, and a cosmetic composition including the enzymatically treated royal jelly extract of Formulation Example 1 was prepared. The composition of the obtained Formulation Example 1 is shown in Table 1.

TABLE 1

| | Proportion |
|---|---|
| Component | (% by weight) |
| Water | 83.79 |
| Butylene glycol | 9.00 |
| Pentylene glycol | 3.00 |
| Enzymatically treated royal jelly extract | 1.50 |
| Glyceryl tri(caprylate/caprate) | 1.50 |
| M Fine oil CCT-1 (MIYOSHI OIL & FAT CO., LTD.) | |
| Phenoxyethanol | 0.60 |
| (Acrylates/(C10-30) alkyl acrylate) crosspolymer | 0.46 |
| (PEMULEN (trademark) TR-1) | |
| Potassium hydroxide | 0.15 |

### Preparation of Cosmetic Composition of Formulation Example 2

The enzymatically treated royal jelly extract, an (ammonium acryloyldimethyltaurate/methacrylic acid beheneth-25) crosspolymer as a skin penetration enhancer, and other raw materials described in Table 2 were mixed at predetermined proportions, and the mixture was stirred at 3000 rpm for 10 minutes by using a homogenizer to prepare a cosmetic composition of Formulation Example 2. The composition of the obtained Formulation Example 2 is shown in Table 2.

TABLE 2

| | Proportion |
|---|---|
| Component | (% by weight) |
| Water | 83.53 |
| Butylene glycol | 9.00 |
| Pentylene glycol | 3.00 |
| Enzymatically treated royal jelly extract | 1.50 |
| Glyceryl tri(caprylate/caprate) | 1.50 |
| (Ammonium acryloyldimethyltaurate/methacrylic acid | 0.86 |
| beheneth-25) crosspolymer | |
| Phenoxyethanol | 0.60 |
| Other regulating agents | Appropriate |
| | amount |

### Preparation of Cosmetic Composition of Comparative Example

The enzymatically treated royal jelly extract and other raw materials described in Table 3 were mixed at predetermined proportions, and a cosmetic composition of Comparative Example was prepared. The composition of the obtained Comparative Example is shown in Table 3.

TABLE 3

| | Proportion |
|---|---|
| Component | (% by weight) |
| Water | 81.28 |
| Butylene glycol | 9.00 |
| Pentylene glycol | 3.00 |
| Enzymatically treated royal jelly extract | 1.50 |
| Phenoxyethanol | 0.60 |
| Glyceryl tri(caprylate/caprate) | 1.50 |
| (Hydroxyethyl acrylate/Sodium acryloyldimethyltaurate) | 1.68 |
| copolymer | |
| Polysorbate 60 | 0.24 |
| Squalane | 1.14 |
| Other regulating agents | Appropriate |
| | amount |

### Skin Penetration Test

The skin penetrability of the cosmetic compositions of Formulation Example 1, Formulation Example 2, and Comparative Example were evaluated by the following method. First, a sample was prepared by a tape stripping test. The forearm part of one volunteer was cleaned and left to stand at room temperature for 15 minutes. Each of the cosmetic compositions of Formulation Example 1, Formulation Example 2, and Comparative Example in an amount of 50 mg/cm² was applied on a square-shaped skin of 6.25 cm² in the forearm part and was left to stand for 2 hours. The applied part was cleaned and left to stand for 15 minutes. Thereafter, a circular-shaped tape (D-Squame sampling discs (CUDERM)) having a diameter of 2 cm was stuck to the applied skin part, the tape was pressed for one minute, subsequently the circular-shaped tape was peeled off, the center part of the circle of the peeled circular-shaped tape was cut into a width of 5 mm, and this was used as a sample for imaging mass spectrometry (imaging MS). Similarly, the operation of sticking a circular-shaped tape to the applied skin part and peeling the tape therefrom was further repeated nine times, and a total of ten samples for imaging MS prepared by cutting the central part of the circle of the circular-shaped tape into a width of 5 mm were obtained. Ten samples as controls were also prepared in the same manner for the skin before applying the cosmetic composition to be tested.

Imaging MS was performed by using DESI Xevo G2 XS QTof (Waters Corporation). The attached amounts of the 10-hydroxy-2-decenoic acid and 10-hydroxydecanoic acid included in royal jelly in each sample were quantitatively determined by detecting m/z 185.1172 and m/z 187.1328 ions.

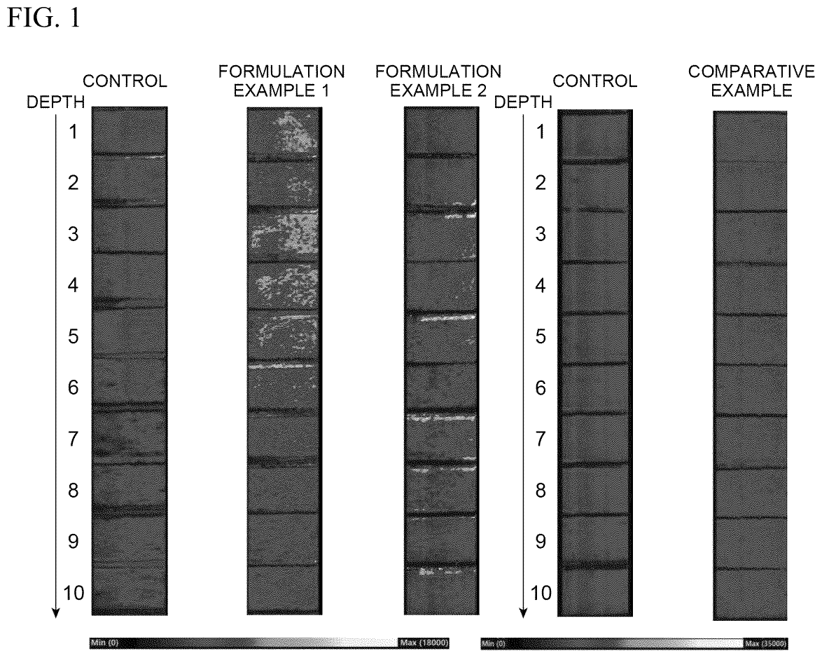

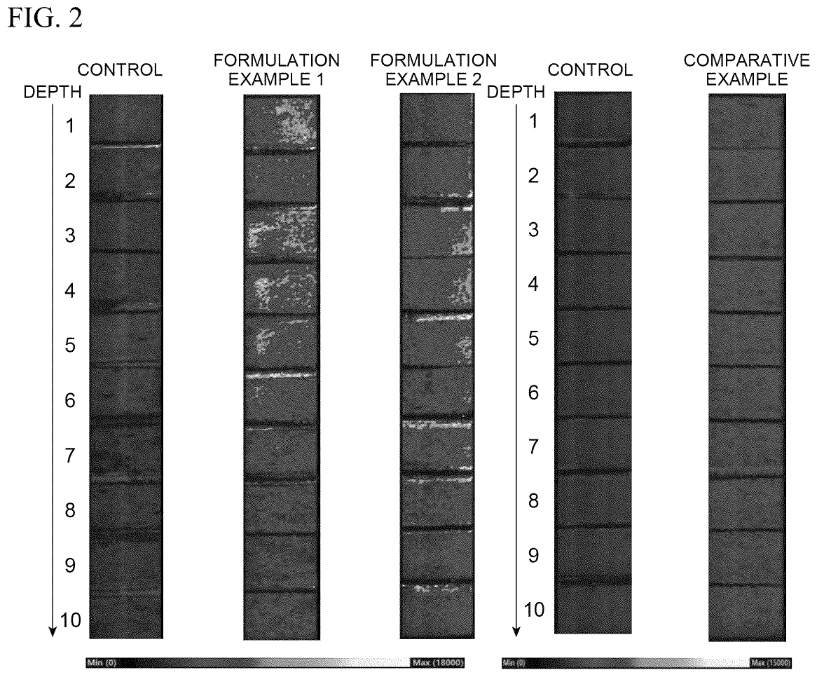

FIG. 1 and FIG. 2 show the results of imaging MS indicating the penetrability of 10-hydroxy-2-decenoic acid and 10-hydroxydecanoic acid into the skin, respectively. From FIG. 1 and FIG. 2, regarding the control and the cosmetic compositions of Comparative Example, the penetration of 10-hydroxy-2-decenoic acid and 10-hydroxydecanoic acid into the skin was not recognized, while 10-hydroxy-2-decenoic acid and 10-hydroxydecanoic acid were detected up to the sixth sheet of tape with the cosmetic composition of Formulation Example 1, and 10-hydroxy-2-decenoic acid and 10-hydroxydecanoic acid were detected up to the fifth sheet of tape with the cosmetic composition of Formulation Example 2. From these, it was proved that the penetration of royal jelly into the skin can be enhanced by adding an (acrylates/(C10-30) alkyl acrylate) crosspolymer or an (ammonium acrylovldimethyltaurate/methacrylic acid beheneth-25) crosspolymer.

What is claimed is:

1. A method for enhancing penetration of a royal jelly into a skin, the method comprising blending an (acrylates/(C10-30) alkyl acrylate) crosspolymer and/or an (ammonium acryloyldimethyltaurate/methacrylic acid beheneth-25) crosspolymer into a cosmetic composition comprising the royal jelly.

2. The method according to claim 1, wherein the royal jelly comprises saturated fatty acids and/or unsaturated fatty acids.

3. The method according to claim 2, wherein the saturated fatty acids comprise 10-hydroxydecanoic acid.

4. The method according to claim 2, wherein the unsaturated fatty acids comprise at least one of 10-hydroxy-2-decenoic acid, decenedioic acid, or sebacic acid.

5. A method for enhancing penetration of a royal jelly into a skin, the method comprising applying a cosmetic composition comprising an (acrylates/(C10-30) alkyl acrylate) crosspolymer and/or an (ammonium acryloyldimethyltaurate/methacrylic acid beheneth-25) crosspolymer and the royal jelly on the skin.

6. The method according to claim 5, wherein the royal jelly comprises saturated fatty acids and/or unsaturated fatty acids.

7. The method according to claim 6, wherein the saturated fatty acids comprise 10-hydroxydecanoic acid.

8. The method according to claim 6, wherein the unsaturated fatty acids comprise at least one of 10-hydroxy-2-decenoic acid, decenedioic acid, or sebacic acid.

9. The method according to claim 5, wherein a content of the (acrylates/(C10-30) alkyl acrylate) crosspolymer in the cosmetic composition is 0.01% by mass or more and 20% by mass or less, with respect to the total amount of the cosmetic composition.

10. The method according to claim 5, wherein a content of the (ammonium acryloyldimethyltaurate/methacrylic acid beheneth-25) crosspolymer in the cosmetic composition is 0.1% by mass or more and 20% by mass or less, with respect to the total amount of the cosmetic composition.

11. The method according to claim 5, wherein a content of the royal jelly in the cosmetic composition is 0.1% by mass or more and 20% by mass or less, with respect to the total amount of the cosmetic composition.

12. A method for producing a cosmetic composition comprising a royal jelly, the method comprising blending an (acrylates/(C10-30) alkyl acrylate) crosspolymer and/or an (ammonium acryloyldimethyltaurate/methacrylic acid beheneth-25) crosspolymer into the cosmetic composition for enhancing penetration of the royal jelly into a skin.

13. The method according to claim 12, wherein the royal jelly comprises saturated fatty acids and/or unsaturated fatty acids.

14. The method according to claim 13, wherein the saturated fatty acids comprise 10-hydroxydecanoic acid.

15. The method according to claim 13, wherein the unsaturated fatty acids comprise at least one of 10-hydroxy-2-decenoic acid, decenedioic acid, or sebacic acid.
